(19) United States
(12) Patent Application Publication
(10) Pub. No.: US 2025/0303083 A1
(43) Pub. Date: Oct. 2, 2025

(54) MEDICAL DEVICE, NON-TRANSITORY STORAGE MEDIUM STORING PROGRAM THEREOF, AND SNORE DETECTING METHOD

(71) Applicant: FUKUDA DENSHI CO., LTD., Tokyo (JP)

(72) Inventors: Ryotaro HASHIMOTO, Tokyo (JP); Mai KOBAYASHI, Tokyo (JP); Tatsuya YONEYAMA, Tokyo (JP)

(73) Assignee: FUKUDA DENSHI CO., LTD., Tokyo (JP)

(21) Appl. No.: 19/089,018

(22) Filed: Mar. 25, 2025

(30) Foreign Application Priority Data
Mar. 29, 2024 (JP) 2024-056650

(51) Int. Cl. A61M 16/00; A61B 5/00

(52) U.S. Cl. CPC A61M 16/024; A61B 5/4818; A61M 2205/3334; A61M 2205/3355; A61M 2205/50

(57) ABSTRACT

A medical device includes a generator configured to generate high frequency data from flow rate data representing a time series of a flow rate of respiration of a patient, a determiner configured to determine, for each of a plurality of periods, an evaluation value of the high frequency data in each period, a selector configured to select one period from among the plurality of periods on the basis of the evaluation values of the plurality of periods, and a detector configured to detect occurrence of a snore of the patient by analyzing a part of the selected one period in the flow rate data.

### BACKGROUND

The present disclosure relates to a medical device, a non-transitory storage medium storing a program thereof, and a snore detecting method.

Nasal continuous positive airway pressure (CPAP) is known as a treatment method for sleep apnea syndrome (SAS). CPAP is a treatment method that suppresses the occurrence of apnea or hypopnea by continuously feeding the airway of a patient with air having appropriate pressure through a nasal mask and thus preventing the obstruction of the airway during a sleep. A snore may occur as a sign of occurrence of apnea or hypopnea. In JP-T-2009-522026, the occurrence of a snore is detected when a value obtained by subtracting a flow rate in an expiration period from a flow rate in an inspiration period exceeds a threshold value.

### SUMMARY

With the method of JP-T-2009-522026, it is difficult to detect a snore with high accuracy. Some aspects of the present disclosure have an object of providing a technology for detecting the occurrence of a snore with high accuracy.

According to some embodiments, there is provided a medical device including generator configured to generate high frequency data from flow rate data representing a time series of a flow rate of respiration of a patient, determiner configured to determine, for each of a plurality of periods, an evaluation value of the high frequency data in each period, selector configured to select one period from among the plurality of periods on the basis of the evaluation values of the plurality of periods, and detector configured to detect occurrence of a snore of the patient by analyzing a part of the selected one period in the flow rate data.

According to some embodiments, it is possible to detect the occurrence of a snore with high accuracy.

### BRIEF DESCRIPTION OF THE DRAWINGS

FIG. 1 is a block diagram of assistance in explaining an example of a configuration of a CPAP device according to some embodiments;

FIG. 2 is a flowchart of assistance in explaining an example of a snore detecting method according to some embodiments;

FIG. 3 is a schematic diagram of assistance in explaining an example of a method for determining an analysis target period according to some embodiments;

FIG. 4 is a schematic diagram of assistance in explaining an example of a detection condition according to some embodiments; and

FIG. 5 is a flowchart of assistance in explaining an example of a treatment pressure changing method according to some embodiments.

### DETAILED DESCRIPTION OF THE PREFERRED EMBODIMENTS

Embodiments will hereinafter be described in detail with reference to the accompanying drawings. It is to be noted that the following embodiments do not limit the disclosure according to claims and that not all of combinations of features described in the embodiments are essential to the disclosure. Two or more features of a plurality of features described in the embodiments may freely be combined with each other. In addition, identical or similar configurations are identified by the same reference numerals, and repeated description thereof will be omitted.

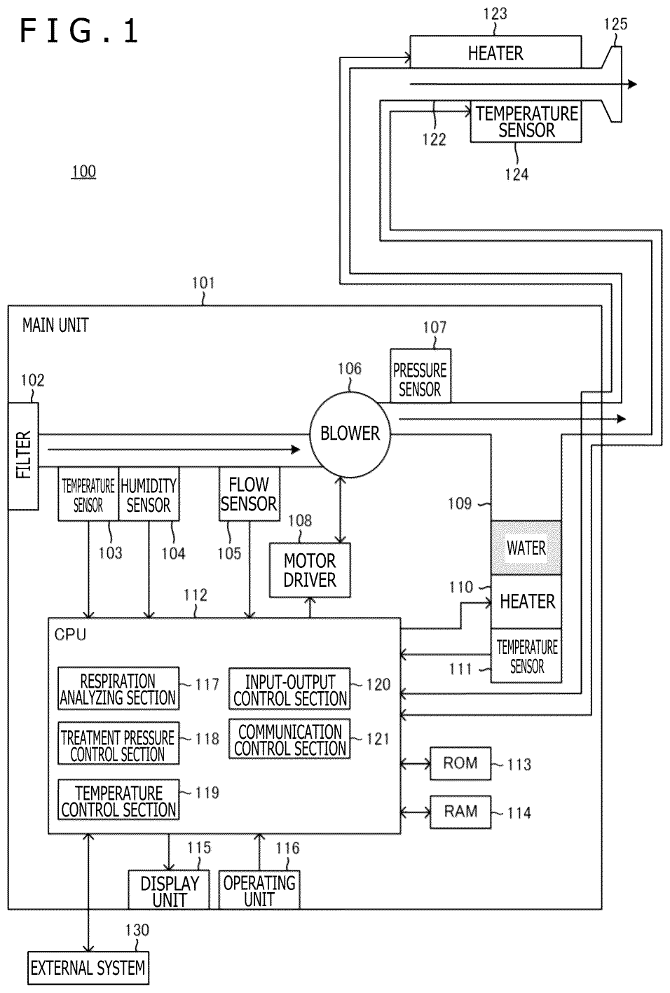

With reference to FIG. 1, description will be made of an example of a configuration of a CPAP device 100 according to some embodiments. The CPAP device 100 is an example of a medical device that has a snore detecting function. The CPAP device 100 includes a main unit 101, a mask 125, and a tube 122 that connects the main unit 101 and the mask 125 to each other. The operation of the CPAP device 100 is implemented by a central processing unit (CPU) 112 reading a program stored in a read only memory (ROM) 113 into a random access memory (RAM) 114 and executing the program. Thus, a device including the CPU 112, the ROM 113, and the RAM 114 may be regarded as a computer. Incidentally, functional blocks 117 through 121 provided within the CPU 112 are schematic representations of main functions of various functions that are implemented by the CPU 112 executing the program. Hence, the operation described with the functional blocks 117 through 121 as entities is actually implemented by the CPU 112 executing the program. Instead of this, one or more functional blocks may be implemented by use of a hardware circuit other than the CPU 112.

Constituent elements present on an air flow passage will first be described. A filter 102 is provided at an air intake port to remove pollen, bacteria, dust, and the like. A temperature sensor 103 measures the temperature of air that flows in. The value measured by the temperature sensor 103 is supplied to a temperature control section 119. A humidity sensor 104 measures the humidity of the air that flows in. The value measured by the humidity sensor 104 is supplied to the temperature control section 119.

A flow (differential pressure) sensor 105 (hereinafter designated simply as a flow sensor 105) is a differential pressure type flow rate sensor, for example. The flow sensor 105 measures the flow rate of the air within the flow passage on the basis of a pressure difference between an upstream side and a downstream side. Suppose in this case that a positive measured value is obtained when pressure on the upstream side is higher than pressure on the downstream side and that a negative measured value is obtained when the pressure on the downstream side is higher than the pressure on the upstream side. This makes it possible also to identify a flow direction of the air within the flow passage from the measured value of the flow sensor 105. The measured value of the flow sensor 105 is supplied to a respiration analyzing section 117.

A blower 106 internally has an impeller and a motor that drives the impeller. A treatment pressure control section 118 controls the rotational speed of the motor through a motor driver 108. The flow rate and supply pressure of the air to be supplied to a patient can thereby be adjusted.

A pressure sensor 107 is provided downstream of the blower 106 on the flow passage. The pressure sensor 107 measures the pressure within the flow passage. The measured value of the pressure sensor 107 is supplied to the treatment pressure control section 118. The treatment pressure control section 118 controls the supply pressure, assuming that the patient is supplied with the air at the pressure measured by the pressure sensor 107.

A humidifier 109 has a water storage tank, and humidifies the air to be supplied to the tube 122. In this case, the temperature control section 119 controls the temperature of a heater 110 provided to the humidifier 109. An amount of water that would be vaporized from the water storage tank, that is, a degree of humidification, is thereby controlled. A temperature sensor 111 measures the temperature of the heater 110 and supplies the temperature of the heater 110 to the temperature control section 119. In the present embodiment, the air to be supplied to the patient is humidified and adjusted in temperature by use of the heater 110 in the humidifier 109. Incidentally, the adjustment of the temperature of the air can be realized also by another method such as the usage of a heater 123 provided to the tube 122, for example. In a case where the humidification and the temperature adjustment are performed separately from each other, the heater 110 and the temperature sensor 111 do not have to be provided to the humidifier 109. In addition, air may be blown onto the surface of water within the water storage tank, or the flow passage may be so disposed as to pass through the water, for example.

The tube 122 connects the main unit 101 and the mask 125 to each other. The tube 122 has elasticity and can easily be bent in order to be able to readily follow movement of the mask 125. The tube 122 is provided with a temperature sensor 124 that measures the temperature of the air supplied to the patient. The measured value of the temperature sensor 124 is supplied to the temperature control section 119.

The mask 125 has such a size and shape as to cover the nose and mouth of the patient. The mask 125 is fitted to the patient by a string or a band that is adjustable in length.

A display unit 115 is, for example, a display provided to a casing of the main unit 101. The display unit 115 displays a message related to the handling of the CPAP device 100, various kinds of menu screens for making settings in the CPAP device 100 and the like, measured values of various kinds of sensors, and the like. The display of the display unit 115 is controlled by an input-output control section 120.

An operating unit 116 is a general term of input devices that can be operated by a user, such as buttons and switches provided to the casing of the main unit 101, for example. In a case where the display unit 115 is a touch display, the display unit 115 and the operating unit 116 are formed integrally with each other. An operation on the operating unit 116 is detected by the input-output control section 120. The CPU 112 performs an action according to the detected operation.

The respiration analyzing section 117 detects the occurrence of a predetermined event on the basis of the flow rate measured by the flow sensor 105. When the respiration analyzing section 117 detects the occurrence of the predetermined event, the respiration analyzing section 117 notifies the treatment pressure control section 118. In the present embodiment, the respiration analyzing section 117 detects the occurrence of a snore of the patient.

The treatment pressure control section 118 controls the operation of the blower 106 such that the supply air pressure becomes a target value, on the basis of the measured value of the pressure sensor 107. In addition, according to a notification from the respiration analyzing section 117, the treatment pressure control section 118 switches between control of the supply air pressure at a time of inspiration of the patient and control of the supply air pressure at a time of expiration of the patient. The treatment pressure control section 118 controls the supply air pressure by providing the motor driver 108 with a duty ratio of a pulsed voltage to be applied to the motor, for example, and thereby controlling the rotational speed of the impeller of the blower 106. The treatment pressure control section 118 notifies the temperature control section 119 of the supply air pressure currently set.

The temperature control section 119 controls the temperature and humidity of the air supplied to the patient, by controlling the operation of the heater 110 according to the measured values of the temperature sensor 103, the humidity sensor 104, the temperature sensor 111, and the temperature sensor 124 and the supply air pressure notified from the treatment pressure control section 118. The temperature and humidity of the air supplied to the patient may be a temperature and a humidity set by the user through the operating unit 116. Through the input-output control section 120, the temperature control section 119 is notified of user settings through the operating unit 116, for example. The supply air pressure is taken into consideration because even when the temperature of the heater 110 is constant, a degree of rise in air temperature is lower in a case of a high flow rate than in a case of a low flow rate.

A communication control section 121 performs processing related to communication between the main unit 101 and an external system 130. The communication control section 121 can perform communication with the external system 130 while complying with one or more of publicly known wireless and/or wire communication standards, for example. The external system 130 may be, for example, a management system for in-hospital medical examination and treatment data or a remote management system for the CPAP device 100.

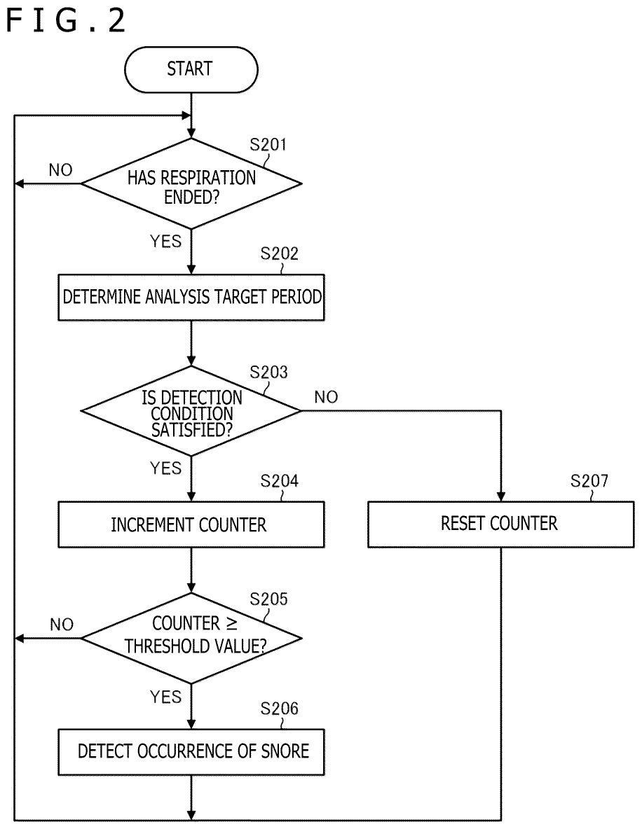

With reference to FIG. 2, description will be made of a method performed by the CPAP device 100 in order to detect a snore. A snore can be a vibration sound generated from the epipharynx when air passes through an airway narrowed during a sleep of the patient. In the following description, each of steps of the method of FIG. 2 is performed by the CPU 112 (for example, the respiration analyzing section 117 of the CPU 112). Specifically, each of the steps is performed by the CPU 112 executing the program read into the RAM 114. Instead of this, at least some of the steps of the method of FIG. 2 may be performed by a dedicated circuit such as an application specific integrated circuit (ASIC). The method of FIG. 2 may be started in response to an instruction given by the patient to start the operation of the CPAP device 100 (for example, treatment operation during the sleep), may be started in response to the detection of falling asleep of the patient by the CPAP device 100, or may be started triggered by another event.

During the execution of the method of FIG. 2, the value measured by the flow sensor 105 continues to be supplied to the CPU 112 (for example, the respiration analyzing section 117 of the CPU 112). As described above, the value measured by the flow sensor 105 represents the flow rate of respiration of the patient. The CPU 112 samples the flow rate supplied by the flow sensor 105 at predetermined sampling intervals (for example, 2 ms) and stores the flow rate at each time as flow rate data in the RAM 114. This flow rate data represents a time series of the flow rate of respiration of the patient. As will be described below in detail, the CPU 112 detects the occurrence of a snore by analyzing the flow rate data.

In S201, the CPU 112 determines whether one present respiration of the patient has ended. When the CPU 112 determines that one present respiration of the patient has ended (“YES” in S201), the CPU 112 shifts the processing to S202. Otherwise (“NO” in S201), the CPU 112 repeats S201. The CPAP device 100 thus waits until one present respiration of the patient ends.

The CPU 112 may detect an end of one present respiration on the basis of the flow rate data. A start point and an end point of one respiration may be set as desired. In the following description, a start of inspiration of the patient is set as a start of one respiration, and an end of expiration of the patient is set as an end of one respiration. In this case, the CPU 112 may detect the end of one respiration on the basis of a change in the flow rate from a value lower than a predetermined threshold value to the threshold value. The predetermined threshold value may be zero, or may be a value (a positive value or a negative value) other than zero. Instead of this, a start of expiration of the patient may be set as the start of one respiration, and an end of inspiration of the patient may be set as the end of one respiration. In this case, the CPU 112 may detect the end of one respiration on the basis of a change in the flow rate from a value higher than the predetermined threshold value to the threshold value. The predetermined threshold value may be zero, or may be a value (a positive value or a negative value) other than zero.

A period during which one respiration is performed will be designated as a respiration period. A period during which an inspiration is performed in the respiration period will be designated as an inspiration period. The inspiration period may be a period during which the flow rate data is higher than the predetermined threshold value (for example, zero or a positive value). A period during which an expiration is performed in the respiration period will be designated as an expiration period. The expiration period may be a period during which the flow rate data is lower than the predetermined threshold value (for example, zero or a negative value).

In S202, the CPU 112 determines a period as a target for analyzing the flow rate data to detect the occurrence of a snore, in one respiration period that has ended most recently. In the following description, the period as a target for analyzing the flow rate data will be designated as an analysis target period. Details of a method for determining the analysis target period will be described later.

In S203, the CPU 112 determines whether a detection condition for detecting a snore is satisfied, by analyzing a part of the analysis target period in the flow rate data. When the CPU 112 determines that the detection condition is satisfied (“YES” in S203), the CPU 112 shifts the processing to S204. Otherwise (“NO” in S203), the CPU 112 shifts the processing to S207. Details of the detection condition will be described later.

In S204, the CPU 112 increments, by one, a counter for counting the number of respirations for which the detection condition is consecutively determined to be satisfied. In the following description, this counter will be designated as a consecutive detection counter. The consecutive detection counter is initialized to zero at a time point of a start in FIG. 2. In S207, the CPU 112 resets the consecutive detection counter to zero.

In S205, the CPU 112 determines whether the value of the consecutive detection counter is equal to or more than a predetermined threshold count. When the CPU 112 determines that the value of the consecutive detection counter is equal to or more than the predetermined threshold count (“YES” in S205), the CPU 112 shifts the processing to S206. Otherwise (“NO” in S205), the CPU 112 shifts the processing to S201.

In S206, the CPU 112 detects that a snore has occurred in one respiration that has ended most recently. At this time, the CPU 112 may store the period of this respiration as a snore occurrence period in the RAM 114. When a snore is detected in a plurality of consecutive respirations, the CPU 112 may store a total period of the plurality of respirations as the snore occurrence period. Instead of this, the CPU 112 may store a total period of a plurality of respirations for which the detection condition is consecutively determined to be satisfied as the snore occurrence period. The snore occurrence period stored in the RAM 114 may be presented to the patient or a doctor after rising of the patient.

The threshold count used in S205 may be one, for example. In this case, the CPU 112 detects the occurrence of a snore when one respiration for which the detection condition is satisfied has occurred. Instead of this, the threshold count used in S205 may be, for example, equal to or more than two (for example, three). In this case, the CPU 112 detects the occurrence of a snore when two or more (for example, three) respirations for which the detection condition is satisfied have occurred consecutively. Detecting the occurrence of a snore when the detection condition is satisfied a plurality of consecutive times in the manner described above makes it possible to suppress an erroneous detection caused by a body movement, a displacement of the mask, or the like.

As described above, in the method of FIG. 2, the CPU 112 performs steps S202 to S206 each time one respiration of the patient is ended. Instead of this, the CPU 112 may perform the steps from S202 on down each time a predetermined number of two or more respirations are ended. For example, the CPU 112 (for example, the respiration analyzing section 117) counts the number of respirations of the patient that are performed after the steps from S202 on down have most recently been performed. The CPU 112 performs the steps from S202 on down when the count value of the number of respirations has reached a predetermined count. The CPU 112 may perform steps S203 through S206 from the oldest respiration in order for each of a predetermined number of most recent respirations. Also in this case, a result similar to that in the case of performing steps S202 through S206 each time one respiration of the patient is ended is obtained. Instead of this modification, the CPU 112 may perform steps S203 through S206 for some of the predetermined number of most recent respirations. For example, the CPU 112 may perform steps S203 through S206 for one most recent respiration among the predetermined number of most recent respirations. In this case, steps S203 through S206 are performed for discrete respirations.

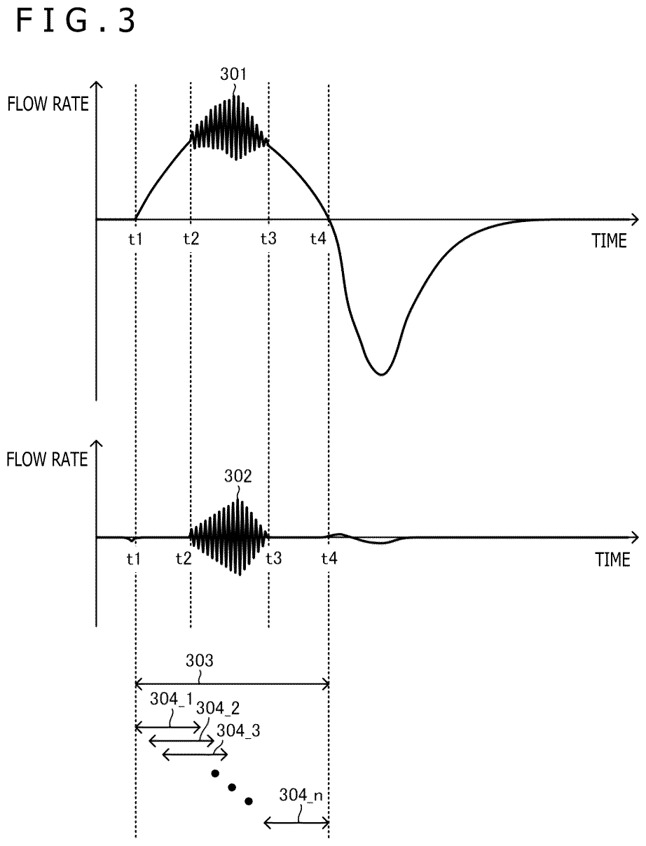

Next, with reference to FIG. 3, description will be made of details of the method for determining the analysis target period in S202 in FIG. 2. A graph 301 in FIG. 3 represents an example of the flow rate data. An axis of abscissa of the graph 301 indicates time. An axis of ordinate of the graph 301 indicates the flow rate. In FIG. 3, the flow rate data is depicted continuously. However, as described above, the flow rate data may be digital data. In the graph 301, focus is placed on the period of one respiration in the flow rate data.

In the example of the graph 301, an inspiration starts at time t1. At time t4, the inspiration ends, and an expiration starts. The graph 301 includes a high-frequency component attributable to a snore from time t2 to t3. Typically, a snore has a frequency of 10 Hz or higher. As a snore occurs, a high-frequency component of 10 Hz or higher is superimposed on the flow rate.

The CPU 112 may determine, as the analysis target period, an inspiration period 303 (time t1 to t4) in one respiration period. It is known that a snore tends to occur not in an expiration but in an inspiration. The expiration can include a noise caused by a disturbance of air due to the exhalation of the patient (for example, a disturbance of air caused by collision between the air supplied from the CPAP device 100 and the expired air of the patient). There is thus a risk of being unable to detect the occurrence of a snore with high accuracy when the occurrence of a snore is to be detected on the basis of a difference between an amount of noise of the inspiration and an amount of noise of the expiration. In the present embodiment, the CPU 112 can detect the occurrence of a snore with high accuracy by including the inspiration period 303 in the analysis target period but not including therein the expiration period.

The CPU 112 may determine the whole of the inspiration period 303 as the analysis target period. Instead of this, the CPU 112 may determine only a part of the inspiration period 303 in which a snore is considered to occur as the analysis target period. It is thereby possible to reduce the calculation cost and improve the accuracy of detection of the occurrence of a snore as compared with the case of setting the whole of the inspiration period 303 as the analysis target period.

In the following, description will be made of a method of determining only a part of the inspiration period 303 as the analysis target period. The CPU 112 applies a filter that cuts off a low-frequency component to the flow rate data. The data generated by this filtering will be designated as high frequency data. The filter that cuts off the low-frequency component may be a high-pass filter, or may be a band-pass filter. For example, the filter that cuts off the low-frequency component may cut off a frequency component lower than a specific frequency (for example, 4.3 Hz) included in a range of 4 to 10 Hz, for example, and pass a frequency component equal to or higher than this specific frequency.

In the case where the flow rate data is digital data, the CPU 112 may perform the filtering by subtracting a moving average of the flow rate data from the flow rate data. For example, the CPU 112 may generate the high frequency data by subtracting a moving average of 51 points from the flow rate data in a case where the sampling intervals of the flow rate data are 2 ms.

A graph 302 of FIG. 3 represents the high frequency data generated by application of a high-pass filter having a cutoff frequency of 4.3 Hz to the flow rate data represented by the graph 301. An axis of abscissa of the graph 302 indicates time. An axis of ordinate of the graph 302 indicates the flow rate.

The CPU 112 may determine the analysis target period on the basis of the high frequency data represented by the graph 302. For example, the CPU 112 may determine, as the analysis target period, a period that has, as a center thereof, a time at which the absolute value of the high frequency data is at a maximum and which has a predetermined time length (that is the time length of a part of the inspiration period 303 of the one respiration, is a value within a range of 400 to 1500 ms, for example, and is 1024 ms, for example). When the period that has the time at which the absolute value of the high frequency data is at a maximum as a center thereof and which has a predetermined time length includes a part not included in the inspiration period 303, the CPU 112 may shift this period such that the whole of this period is included in the inspiration period 303. At this time, the CPU 112 may shift the analysis target period while maintaining the predetermined time length such that a start point of the analysis target period coincides with a start point of the inspiration period 303 or such that an end point of the analysis target period coincides with an end point of the inspiration period 303. In addition, the CPU 112 may change (shorten or lengthen) the analysis target period including a part located before the start point of the inspiration period 303 or the analysis target period including a part after the end point of the inspiration period 303 without maintaining the predetermined time length such that the start point of the analysis target period coincides with the start point of the inspiration period 303 or such that the end point of the analysis target period coincides with the end point of the inspiration period 303.

Instead of this, the CPU 112 may determine, as the analysis target period, one candidate period among a plurality of candidate periods 304_1 through 304_n included in the inspiration period 303 of one respiration. The plurality of candidate periods 304_1 through 304_n will be designated collectively as candidate periods 304. The following description of one candidate period 304 applies also to any of the plurality of candidate periods 304_1 through 304_n. A suffix of a candidate period 304 has a value that is smaller as a start point of the candidate period 304 is closer to the start point of the inspiration period 303 (that is, as the start point of the candidate period 304 is an older time). In the following, the plurality of candidate periods 304_1 through 304_n will be designated simply as a plurality of candidate periods 304.

In the example of FIG. 3, the plurality of candidate periods 304 having the same time length are so arranged as to be shifted from one another by the same interval in such a manner as to cover the whole of the inspiration period 303. In other words, each of the plurality of candidate periods 304 has the same time length. The time length of the candidate periods 304 may be a value within a range of 400 to 1500 ms, for example, and may be 1024 ms, for example. Start points of the plurality of candidate periods 304 are arranged at the same time intervals. The intervals may be wider than the sampling intervals of the flow rate data (for example, 2 ms), or may be equal to the sampling intervals. The intervals may be a value within a range of 30 to 150 ms, for example, and may be 64 ms, for example. When the intervals are too short, the number of candidate periods 304 is increased, and consequently, the calculation cost of processing to be described later becomes high. When the intervals are too long, on the other hand, the number of candidate periods 304 becomes small, and choices for the analysis target period are reduced. The intervals of the start points of the plurality of candidate periods 304 may be a divisor of the analysis target period described above. End points of the plurality of candidate periods 304 are also arranged at the same time intervals. The start point of the first candidate period 304_1 coincides with the start point of the inspiration period 303. Instead of this, the start point of the first candidate period 304_1 may be after the start point of the inspiration period 303. The end point of the last candidate period 304_n coincides with the end point of the inspiration period 303. Instead of this, the end point of the last candidate period 304_n may be before the end point of the inspiration period 303.

In the example of FIG. 3, the intervals of the plurality of candidate periods 304 are shorter than the time length of one candidate period 304. Hence, each of the plurality of candidate periods 304 partly overlaps another candidate period of the plurality of candidate periods 304. For example, the candidate period 304_1 partly overlaps the candidate periods 304_2, 304_3, and so on. It is thus possible to include more candidate periods 304 in the inspiration period 303, and consequently analyze the inspiration period 303 with high accuracy. Instead of this, the intervals of the plurality of candidate periods 304 may be equal to the time length of one candidate period 304 or may be longer than the time length of one candidate period 304. In this case, each of the plurality of candidate periods 304 does not overlap another candidate period of the plurality of candidate periods 304.

Instead of the example of FIG. 3, the plurality of candidate periods 304 may have different time lengths. In addition, the start points of the plurality of candidate periods 304 do not have to be arranged at the same time intervals. It is considered that a snore is more likely to occur near the middle of an inspiration than an end of the inspiration. Accordingly, for example, short candidate periods 304 may be arranged more densely in the vicinity of the center of the analysis target period than in the end part of the analysis target period.

In the above-described example, the plurality of candidate periods 304 are included in the inspiration period 303 of one respiration. Instead of this, the plurality of candidate periods 304 may include a part not included in the inspiration period 303 of one respiration. For example, the plurality of candidate periods 304 may include a candidate period 304 included in the expiration period. Further, the plurality of candidate periods 304 may be distributed over the inspiration periods 303 of a plurality of respirations.

Next, description will be made of a method of selecting one candidate period 304 that becomes the analysis target period from the plurality of candidate periods 304. For each of the plurality of candidate periods 304, the CPU 112 determines an evaluation value of the high frequency data in each of the candidate periods 304. In the following, the evaluation value of the high frequency data in one candidate period will be designated simply as the evaluation value of the candidate period. The evaluation value may be a value for evaluating a likelihood of the occurrence of a snore of the patient in one candidate period 304. Specifically, the evaluation value of one candidate period 304 is a value increased as a possibility of the occurrence of a snore of the patient in the candidate period 304 is increased.

The CPU 112 may determine a maximum value of the absolute value of the high frequency data in one candidate period 304 as the evaluation value of the candidate period 304. Instead of this, the CPU 112 may determine the evaluation value on the basis of an integrated value of the absolute value of the high frequency data in one candidate period 304. Specifically, the CPU 112 may determine, as the evaluation value, an integrated value of the absolute value of the high frequency data in one candidate period 304. It is known that a snore continues for a certain period of time during one inspiration. It is hence possible to detect the occurrence of a snore with high accuracy by determining the evaluation value on the basis of the integrated value of the absolute value of the high frequency data. In a case where the plurality of candidate periods 304 have different time lengths, a value obtained by dividing the integrated value of the absolute value of the high frequency data by the time length of the candidate period 304 may be determined as the evaluation value.

Description will be made of an example of a method for determining the evaluation value of the high frequency data for each of the plurality of candidate periods 304 in the case where the integrated value of the absolute value of the high frequency data in one candidate period 304 is determined as the evaluation value. First, the CPU 112 obtains the integrated value of the absolute value of the high frequency data for the candidate period 304_1. This integrated value is the evaluation value of the candidate period 304_1. The CPU 112 thereafter obtains the integrated value of the absolute value of the high frequency data from the start point of the candidate period 304_1 to the start point of the candidate period 304_2 (which will hereinafter be designated as a start point integrated value) and the integrated value of the absolute value of the high frequency data from the end point of the candidate period 304_1 to the end point of the candidate period 304_2 (which will hereinafter be designated as an end point integrated value). The CPU 112 thereafter determines the evaluation value of the candidate period 304_2 by subtracting the start point integrated value from the evaluation value of the candidate period 304_1 and adding the end point integrated value. The CPU 112 thereafter similarly determines the evaluation value of each of the candidate periods 304_3 through 304_n. Instead of the method of calculating only the differences as described above, the CPU 112 may individually obtain the integrated value of the absolute value of the high frequency data for each of the plurality of candidate periods 304.

The CPU 112 selects one candidate period from among the plurality of candidate periods 304 by comparing the evaluation values of the plurality of candidate periods 304 with each other after determining the evaluation value of each of the plurality of candidate periods 304.

In one example, the CPU 112 may select one candidate period 304 having the maximum evaluation value among the plurality of candidate periods 304. When there are a plurality of candidate periods 304 having the maximum evaluation value, the CPU 112 may select one candidate period 304 located at the center on a time axis among these plurality of candidate periods 304. In another example, the CPU 112 may select a predetermined number of (for example, five) candidate periods 304 in descending order of the evaluation values from among the plurality of candidate periods 304, and select one candidate period 304 located at the center on the time axis among the predetermined number of candidate periods 304.

Instead of the above-described method, the CPU 112 may select one candidate period from among the plurality of candidate periods 304 without comparing the evaluation values of the plurality of candidate periods 304 with each other. For example, the CPU 112 may identify one or more candidate periods 304 having an evaluation value exceeding a predetermined threshold value among the plurality of candidate periods 304, and freely select one candidate period 304 from the one or more candidate periods 304. When none of the evaluation values of the plurality of candidate periods 304 exceeds the predetermined threshold value, the CPU 112 may determine that no snore has occurred in this respiration.

The detection condition in S203 in FIG. 2 will next be described. In one example, the detection condition may be that the above-described evaluation value of the analysis target period is equal to or more than a predetermined threshold value. In another example, the detection condition may be that a result of frequency analysis of the flow rate data in the analysis target period satisfies a predetermined condition. The occurrence of a snore can be detected with high accuracy when the occurrence of the snore is detected by use of the result of the frequency analysis in the manner described above.

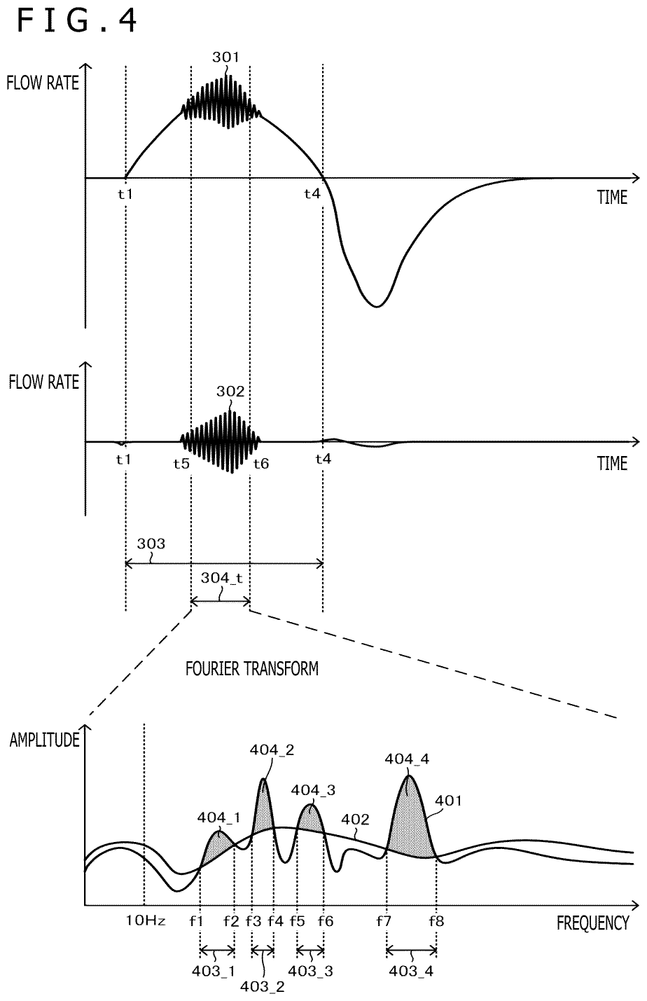

With reference to FIG. 4, description will be made of an example of frequency analysis of the flow rate data. Graphs 301 and 302 in FIG. 4 are the same as the graphs 301 and 302 in FIG. 3. Suppose that a period 304_t from time t5 to time t6 is determined as the analysis target period as a result of S202 in FIG. 2.

The CPU 112 generates frequency data by performing signal processing including a Fourier transform on the part of the period 304_t in the flow rate data. This signal processing may include filtering before the Fourier transform. For example, the CPU 112 may directly perform the Fourier transform on the part of the period 304_t in the flow rate data. Instead of this, the CPU 112 may generate high frequency data by applying a filter for cutting off a low-frequency component to the flow rate data, and perform the Fourier transform on the part of the period 304_t in the high frequency data. The Fourier transform may be performed by a fast Fourier transform (FFT).

A graph 401 of FIG. 4 represents the frequency data generated by the Fourier transform. An axis of abscissa of the graph 401 indicates frequency. An axis of ordinate of the graph 401 indicates spectrum amplitude. The detection condition may be a condition related to the frequency data and reference data. For example, the detection condition may be a condition related to a relation between the frequency data and the reference data. The reference data is data having a frequency distribution, and has a frequency distribution that can be assumed by the frequency data when no snore is presumed to have occurred. A graph 402 of FIG. 4 represents the reference data.

The reference data may be stored in the ROM 113 before a start of the method of FIG. 2 (for example, before a start of a sleep of the patient). For example, a manufacturer of the CPAP device 100 may generate the reference data from a plurality of subjects on the basis of the flow rate data of respiration during a sleep in a state in which no snore is occurring, and store the reference data in the ROM 113 of the CPAP device 100. For example, the manufacturer may generate a frequency spectrum by Fourier transforming the flow rate data (or the high frequency data thereof) of each respiration of the plurality of subjects, and set, as the reference data, a value obtained by adding a double standard deviation to an average of the frequency spectrum.

Instead of this, the reference data may be generated during a sleep of the patient and stored in the RAM 114. A possibility of the occurrence of a snore is considered to be low immediately after the patient falls asleep. Accordingly, the CPU 112 may generate a frequency spectrum by Fourier transform of the flow rate data (or the high frequency data thereof) of each respiration of the patient for a predetermined period of time (for example, for 15 minutes or for 30 minutes) from the falling asleep, and set, as the reference data, a value obtained by adding a double standard deviation to an average of the frequency spectrum.

The detection condition may include a condition related to an integrated value of a difference between the frequency data and the reference data in one or more bands in which the frequency data exceeds the reference data in a specific frequency band. This condition will be designated as an integrated value condition. The specific frequency band in the integrated value condition may be a frequency band including the vibration of air caused by a snore, and may be equal to or higher than 10 Hz, for example. An upper limit of the specific frequency band may not exist, or may be 100 Hz or 200 Hz. In addition, the specific frequency band may have an upper limit determined by a sampling theorem. For example, the upper limit of the frequency band in a case where the flow rate data is sampled at 500 Hz is 250 Hz.

In the example of FIG. 4, in the frequency band of 10 Hz and higher, the frequency data exceeds the reference data in four bands 403_1 through 403_4. The plurality of bands 403_1 through 403_4 will be designated collectively as bands 403. The following description of one band 403 applies also to any of the plurality of bands 403_1 through 403_4. In the following, the plurality of bands 403_1 through 403_4 will be designated simply as a plurality of bands 403.

The integrated value of the difference between the frequency data and the reference data in the plurality of bands 403 represents a sum of the areas of regions 404_1 through 404_4. The integrated value condition may include the above-described integrated value being equal to or more than a predetermined threshold value. This threshold value may be stored in the ROM 113 before a start of the method of FIG. 2 (for example, during the manufacturing of the CPAP device 100). It is known that the frequencies of a snore are distributed in such a manner as to spread over a certain width. The occurrence of a snore can hence be detected with high accuracy when the detection condition includes the integrated value condition.

The detection condition may include a condition related to a relative value of the frequency data with respect to the reference data in one of one or more bands in which the frequency data exceeds the reference data in the specific frequency band. The relative value in each of the one or more bands may be a maximum value of a difference of the frequency data with respect to the reference data (that is, a value obtained by subtracting the reference data from the frequency data) in one band, or may be a maximum value of a ratio of the frequency data with respect to the reference data (that is, a value obtained by dividing the frequency data by the reference data) in this one band.

The detection condition may include a condition related to the relative value in one of the one or more bands in which the frequency data exceeds the reference data in a specific frequency band. This condition will be designated as a primary relative value condition. The specific frequency band in the primary relative value condition may be the same as the specific frequency band in the integrated value condition.

The one band used in the primary relative value condition may be a band in which the relative value is at a maximum among the one or more bands in which the frequency data exceeds the reference data in the specific frequency band. For example, the band 403_4 among the plurality of bands 403 may be used in the primary relative value condition. The primary relative value condition may include the relative value in this band being equal to or more than a predetermined threshold value. This threshold value may be stored in the ROM 113 before a start of the method of FIG. 2 (for example, during the manufacturing of the CPAP device 100).

The detection condition may include a condition related to the relative value in one band that is different from the band used in the primary relative value condition and that is among the one or more bands in which the frequency data exceeds the reference data in the specific frequency band. This condition will be designated as a secondary relative value condition. The specific frequency band in the secondary relative value condition may be the same as the specific frequency band in the integrated value condition.

The one band used in the secondary relative value condition may be a band in which the relative value is the second largest among the one or more bands in which the frequency data exceeds the reference data in the specific frequency band. For example, the band 403_2 among the plurality of bands 403 may be used in the secondary relative value condition. The secondary relative value condition may include the relative value in this band being equal to or more than a predetermined threshold value. This threshold value may be stored in the ROM 113 before a start of the method of FIG. 2 (for example, during the manufacturing of the CPAP device 100).

The CPU 112 may use only one of the integrated value condition, the primary relative value condition, and the secondary relative value condition as the detection condition, or may use a combination of two or more of these three conditions as the detection condition. For example, the CPU 112 may determine whether each of the integrated value condition, the primary relative value condition, and the secondary relative value condition is satisfied, and determine that the detection condition is satisfied, on the basis of satisfaction of at least one of these three conditions. For example, the CPU 112 may determine whether each of the integrated value condition and the primary relative value condition is satisfied, and determine that the detection condition is satisfied, on the basis of satisfaction of at least one of these two conditions.

In the determination of S203 in FIG. 2, the CPU 112 may adjust the value of the reference data, and determine whether the detection condition is satisfied, by using the reference data available after the adjustment. It is known that the magnitude (that is, the spectrum amplitude) of the frequency data is proportional to the flow rate. Accordingly, the CPU 112 may adjust the value of the reference data on the basis of the flow rate of an inspiration of each respiration.

For example, the CPU 112 determines a representative value of the flow rate data in the inspiration period of each respiration. The representative value may be a maximum value of the flow rate data in the inspiration period, may be an average value of the flow rate data in the inspiration period, may be an integrated value of the flow rate data over the inspiration period, or may be another value. A reference flow rate is stored in association with the reference data in the ROM 113 or the RAM 114. The CPU 112 may adjust the value of the reference data by multiplying the reference data (that is, multiplying the spectrum amplitude at each frequency of the reference data) by a value obtained by dividing the representative value of the flow rate data in the inspiration period of each respiration by the reference flow rate.

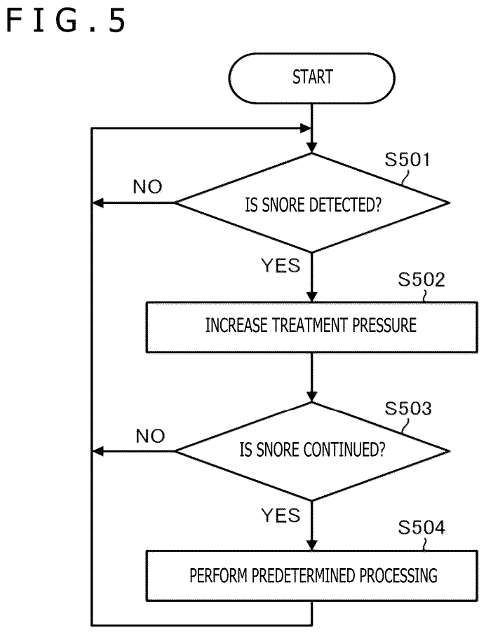

With reference to FIG. 5, description will be made of a method performed by the CPAP device 100 to adjust treatment pressure. The detection of the occurrence of a snore may be used to adjust the temperature and the humidity provided to the patient, or may be used for other processing, in addition to or in place of the adjustment of the treatment pressure. In the following description, each of steps of the method of FIG. 5 is performed by the CPU 112 (for example, the treatment pressure control section 118 of the CPU 112). Specifically, each of the steps is performed by the CPU 112 executing the program read into the RAM 114. Instead of this, at least some of the steps of the method of FIG. 5 may be performed by a dedicated circuit such as an ASIC. The method of FIG. 5 may be started in response to an instruction given by the patient to start the operation of the CPAP device 100 (for example, treatment operation during a sleep), may be started in response to the detection of falling asleep of the patient by the CPAP device 100, or may be started triggered by another event. In the method of FIG. 5, the treatment pressure is changed in response to the detection of a snore. In addition to this, the treatment pressure may be changed in response to the detection of another event (for example, the passage of a predetermined period of time or the occurrence of apnea).

In S501, the CPU 112 determines whether the occurrence of a snore is detected. When the CPU 112 determines that the occurrence of a snore is detected (“YES” in S501), the CPU 112 shifts the processing to S502. Otherwise (“NO” in S501), the CPU 112 repeats S501. The CPAP device 100 thus waits until the occurrence of a snore is detected. The occurrence of a snore of the patient is detected in real time during a sleep of the patient.

In S502, the CPU 112 increases the present treatment pressure by a predetermined value or ratio. The treatment pressure may be the pressure of the air supplied to the patient.

In S503, the CPU 112 determines whether the snore is continued. When the CPU 112 determines that the snore is continued (“YES” in S503), the CPU 112 shifts the processing to S504. Otherwise (“NO” in S503), the CPU 112 shifts the processing to S501.

The obstruction of the airway is possibly resolved by an increase in the treatment pressure when the snore is not continued (that is, when the snore is stopped). The CPU 112 hence maintains this treatment pressure. Meanwhile, the snore is possibly not caused by the obstruction of the airway when the snore is continued even with an increase in the treatment pressure. Accordingly, in S504, the CPU 112 performs predetermined processing. For example, the CPU 112 may decrease the treatment pressure. For example, the CPU 112 may turn the treatment pressure back to a value used before the treatment pressure is increased in S502. The CPU 112 may output an alert indicating that a snore not caused by the obstruction of the airway has occurred, in addition to or in place of the decreasing of the treatment pressure. An output destination of the alert may be the doctor or the like. A snore not caused by the obstruction of the airway can occur due to nasal congestion, oral respiration, or the like. The doctor or the like may give an instruction for the nasal congestion or the wearing of the mask on the basis of the alert.

The treatment pressure may have an upper limit value set by the doctor, for example. In a case where the treatment pressure has reached the upper limit value, the CPU 112 does not have to increase the treatment pressure even when the occurrence of a snore is detected in S501. The treatment pressure may be reset to an initial value in each treatment (for example, each day). This initial value may be a minimum treatment pressure set by the doctor, for example. In addition, the CPU 112 may decrease the treatment pressure when an event of a respiration disorder such as a snore, apnea, or hypopnea is not detected for a certain period of time or more.

In the foregoing embodiment, the snore detecting method of FIG. 2 is performed by the CPAP device 100. Instead of this, the snore detecting method of FIG. 2 may be performed by a testing apparatus for SAS. Such a testing apparatus does not have a function of supplying compressed air to the patient. The SAS testing apparatus that performs the snore detecting method of FIG. 2 is also a medical device having the snore detecting function.

In the foregoing embodiment, the occurrence of a snore of the patient is detected during a sleep of the patient. Instead of this, the snore detecting method of FIG. 2 may be performed after the rising of the patient with use of the flow rate data obtained during a sleep of the patient. That is, the occurrence of a snore of the patient may be detected after the rising of the patient. A period of the occurrence of the snore can thus be identified, and can be used for the diagnosis of the patient.

### SUMMARY OF EMBODIMENTS

### [Item 1]

A medical device including:
generator configured to generate high frequency data from flow rate data representing a time series of a flow rate of respiration of a patient;
determiner configured to determine, for each of a plurality of periods, an evaluation value of the high frequency data in each period;
selector configured to select one period from among the plurality of periods on the basis of the evaluation values of the plurality of periods; and
detector configured to detect occurrence of a snore of the patient by analyzing a part of the selected one period in the flow rate data.

According to this item, the flow rate data is analyzed for the selected period, and the occurrence of a snore can thus be detected with high accuracy.

### [Item 2]

The medical device according to item 1, in which
the selector selects the one period from among the plurality of periods by comparing the evaluation values of the plurality of periods with each other.

According to this item, an appropriate period can be selected by comparing the evaluation values with each other, and the occurrence of a snore can thus be detected with high accuracy.

### [Item 3]

The medical device according to item 1 or 2, in which
the plurality of periods are included in an inspiration period.

According to this item, the occurrence of a snore can be detected with high accuracy by analyzing the flow rate data in the inspiration period in which a snore is highly likely to be included.

### [Item 4]

The medical device according to any one of items 1 through 3, in which
each of the plurality of periods partly overlaps another period of the plurality of periods.

According to this item, the number of the plurality of periods in a certain time length can be increased, and a plurality of evaluation values can thus be determined with fine granularity.

### [Item 5]

The medical device according to any one of items 1 through 4, in which
the plurality of periods each have the same time length.

According to this item, the evaluation values can be determined on the same scale.

### [Item 6]

The medical device according to any one of items 1 through 5, in which
start points of the plurality of periods are arranged at same time intervals.

According to this item, the plurality of periods can be selected from a target period in a well-balanced manner.

### [Item 7]

The medical device according to item 6, in which
the flow rate data is digital data, and
an interval between the start points of the plurality of periods is wider than a sampling interval of the flow rate data.

According to this item, a processing load for detecting the occurrence of a snore can be reduced.

### [Item 8]

The medical device according to any one of items 1 through 7, in which
the evaluation value has a value that is larger as a possibility of the occurrence of a snore is increased, and
the selector selects the one period in which the evaluation value is at a maximum from among the plurality of periods.

According to this item, the period in which there is a high possibility of the occurrence of a snore can be selected with high accuracy.

### [Item 9]

The medical device according to any one of items 1 through 8, in which
the determiner determines the evaluation value on the basis of an integrated value of an absolute value of the high frequency data in each period.

According to this item, the evaluation value can be determined with high accuracy on the basis of such a characteristic of a snore that the snore continues for a certain period of time.

### [Item 10]

The medical device according to item 1, in which
the detector detects the occurrence of the snore of the patient in real time during a sleep of the patient.

According to this item, a treatment for the snore can be performed in real time during a sleep.

### [Item 11]

The medical device according to item 10, in which
the medical device is a continuous positive airway pressure device, and
the medical device further includes pressure controller configured to increase pressure of air supplied to the patient, on the basis of detection of the occurrence of the snore of the patient.

According to this item, the occurrence of a respiration disorder can be prevented.

### [Item 12]

A program or a non-transitory storage medium storing a program for making a computer to execute processing comprising:
generating high frequency data from flow rate data representing a time series of a flow rate of respiration of a patient;
determining, for each of a plurality of periods, an evaluation value of the high frequency data in each period;
selecting one period from among the plurality of periods on a basis of the evaluation values of the plurality of periods; and
detecting occurrence of a snore of the patient by analyzing a part of the selected one period in the flow rate data.

According to this item, a program or a non-transitory storage medium storing a program for implementing the medical device having the above-described effects is provided.

### [Item 13]

A snore detecting method performed by a computer, the snore detecting method including:
generating high frequency data from flow rate data representing a time series of a flow rate of respiration of a patient;
determining, for each of a plurality of periods, an evaluation value of the high frequency data in each period;
selecting one period from among the plurality of periods on the basis of the evaluation values of the plurality of periods; and
detecting occurrence of a snore of the patient by analyzing a part of the selected one period in the flow rate data.

According to this item, the flow rate data is analyzed for the selected period, and the occurrence of a snore can thus be detected with high accuracy.

The disclosure is not limited to the foregoing embodiments, and is susceptible of various modifications and changes within the spirit of the disclosure.

What is claimed is:

1. A medical device comprising:
generator configured to generate high frequency data from flow rate data representing a time series of a flow rate of respiration of a patient;
determiner configured to determine, for each of a plurality of periods, an evaluation value of the high frequency data in each period;
selector configured to select one period from among the plurality of periods on a basis of the evaluation values of the plurality of periods; and
detector configured to detect occurrence of a snore of the patient by analyzing a part of the selected one period in the flow rate data.

2. The medical device according to claim 1, wherein
the selector selects the one period from among the plurality of periods by comparing the evaluation values of the plurality of periods with each other.

3. The medical device according to claim 1, wherein
the plurality of periods are included in an inspiration period.

4. The medical device according to claim 1, wherein
each of the plurality of periods partly overlaps another period of the plurality of periods.

5. The medical device according to claim 1, wherein
the plurality of periods each have a same time length.

6. The medical device according to claim 1, wherein
start points of the plurality of periods are arranged at same time intervals.

7. The medical device according to claim 6, wherein
the flow rate data is digital data, and
an interval between the start points of the plurality of periods is wider than a sampling interval of the flow rate data.

8. The medical device according to claim 1, wherein
the evaluation value has a value that is larger as a possibility of the occurrence of a snore is increased, and
the selector selects the one period in which the evaluation value is at a maximum from among the plurality of periods.

9. The medical device according to claim 1, wherein
the determiner determines the evaluation value on a basis of an integrated value of an absolute value of the high frequency data in each period.

10. The medical device according to claim 1, wherein
the detector detects the occurrence of the snore of the patient in real time during a sleep of the patient.

11. The medical device according to claim 10, wherein
the medical device is a continuous positive airway pressure device, and
the medical device further includes pressure controller configured to increase pressure of air supplied to the patient, on a basis of detection of the occurrence of the snore of the patient.

12. A non-transitory storage medium storing a program for making a computer to execute processing comprising:
generating high frequency data from flow rate data representing a time series of a flow rate of respiration of a patient;
determining, for each of a plurality of periods, an evaluation value of the high frequency data in each period;
selecting one period from among the plurality of periods on a basis of the evaluation values of the plurality of periods; and
detecting occurrence of a snore of the patient by analyzing a part of the selected one period in the flow rate data.

13. A snore detecting method performed by a computer, the snore detecting method comprising:
generating high frequency data from flow rate data representing a time series of a flow rate of respiration of a patient;
determining, for each of a plurality of periods, an evaluation value of the high frequency data in each period;
selecting one period from among the plurality of periods on a basis of the evaluation values of the plurality of periods; and
detecting occurrence of a snore of the patient by analyzing a part of the selected one period in the flow rate data.
